(19) United States
(12) Patent Application Publication
(10) Pub. No.: US 2016/0206367 A1
(43) Pub. Date: Jul. 21, 2016

(54) ELECTROSURGICAL INSTRUMENT

(71) Applicant: GYRUS MEDICAL LIMITED, Cardiff (GB)

(72) Inventor: Kelvin John VARNEY, Abergavenny (GB)

(21) Appl. No.: 14/994,536

(22) Filed: Jan. 13, 2016

(30) Foreign Application Priority Data
Jan. 16, 2015 (GB) 1500716.4

(51) Int. Cl. A61B 18/14

(52) U.S. Cl. CPC A61B 18/1445; A61B 2018/00077

(57) ABSTRACT

An electrosurgical instrument includes a pair of opposing first and second jaw members (5), (6) movable from a first open position in which the jaw members are disposed in a spaced relation relative to one another, to a second closed position in which the jaw members cooperate to grasp tissue therebetween. A first sealing electrode (7) is located on the first jaw member, and a second sealing electrode (8) is located on the second jaw member. A stationary flexible cutting electrode (11) is located adjacent the throat area constituted by the convergence of the first and second jaw members when in their open position. The electrical connections of the instrument are such that when the jaw members (5), (6) are in their closed position with tissue grasped therebetween, the instrument is capable of sealing the tissue by passing an electrosurgical current into the tissue from the first and second sealing electrodes (7), (8). When the jaw members (5), (6) are in their open position the instrument is capable of cutting tissue by moving the instrument in a forward direction such that tissue comes into contact with the cutting electrode (11), an electrosurgical current passing into the tissue from the cutting electrode.

### TECHNICAL FIELD

Embodiments of this invention relate to an electrosurgical instrument for cutting tissue, and to a method for cutting tissue using an electrosurgical instrument and an associated electrosurgical generator. Such instruments are commonly used for the treatment of tissue in surgical intervention, most commonly in “keyhole” or minimally invasive surgery, but also in “open” surgery.

### BACKGROUND TO THE INVENTION AND PRIOR ART

It is known to provide an electrosurgical instrument in which the cutting of tissue is effected by means of an elongate electrosurgical electrode extending along the inner surface of one of a pair of jaw elements. U.S. Pat. No. 6,174,309 & 7,204,835 are two examples of this kind of instrument. U.S. Pat. No. 7,204,835 provides an arrangement in which tissue may be cut by the forward movement of a jawed instrument with the jaws held in an open position, a so-called “running cut”.

### SUMMARY OF THE INVENTION

Embodiments of the present invention attempt to provide an improvement to electrosurgical instruments such as those described above.

Accordingly, an electrosurgical instrument is provided including a handle including an actuating mechanism movable between a first position and a second position,
a pair of opposing first and second jaw members, movement of the actuating mechanism from its first position to its second position causing at least one of the jaw members to move relative to the other from a first open position in which the jaw members are disposed in a spaced relation relative to one another, to a second closed position in which the jaw members cooperate to grasp tissue therebetween, the first and second jaw members defining a throat area at which the jaw members converge when in their open position,
a first sealing electrode located on the first jaw member,
a second sealing electrode located on the second jaw member,
a flexible cutting electrode located adjacent the throat area and in fixed relationship thereto, and
electrical connections capable of connecting the instrument to a source of electrosurgical current, such that when the jaw members are in their closed position with tissue grasped therebetween, the instrument is capable of sealing the tissue by passing an electrosurgical current into the tissue from the first and second sealing electrodes, and when the jaw members are in their open position the instrument is capable of cutting tissue by moving the instrument in a forward direction such that tissue comes into contact with the cutting electrode, an electrosurgical current passing into the tissue from the cutting electrode.

The fixed cutting electrode located adjacent the throat area is specifically designed for use in a “running cut”, in which the first and second jaw members are held partially open, and the instrument is moved in a forward direction against tissue entering the V-shaped gap between the first and second jaw members. With a cutting RF waveform supplied to the cutting electrode, the tissue is severed in a “running cut” in a manner similar to moving open scissors forwardly through wrapping paper. Published US patent application 2013/0006242 describes a forceps instrument with a rigid bar as a blade electrode positioned between the jaws. In contrast, embodiments of the present invention provide a stationary yet flexible electrode adjacent the throat area of the instrument.

The source of electrosurgical current is conveniently an external electrosurgical generator, although it is conceivable that the electrosurgical generator may be contained within the instrument itself as a self-contained “cordless” instrument. In either arrangement, the connections are capable of directing an electrosurgical current to the relevant electrodes carried by the instrument.

The flexible cutting electrode preferably comprises a wire extending from the first jaw member to the second jaw member. Typically, the wire is located on the longitudinal axis on the centre line of the first and second jaw members. Alternatively, the cutting electrode conceivably comprises two wires extending from the first jaw member to the second jaw member, one on the outer face of each of the first and second jaw members. By the term “wire” or “wires” there is herein meant to include a flexible elongate metallic strip of whatever cross-section, not just those having a circular cross-section. Whichever arrangement is employed, tissue comes into contact with the cutting electrode as the instrument is advanced through tissue with the jaw members held partially open, and the tissue is severed by the electrosurgical current supplied to the cutting electrode.

The first and second jaw members are preferably pivotable with respect to one another about a pivot mechanism, and the cutting electrode is conveniently adjacent the pivot mechanism. The handle conveniently includes first and second pivoting arms, a distal portion of the first arm constituting the first jaw member and a distal portion of the second arm constituting the second jaw member. The first sealing electrode is preferably electrically insulated from the first jaw member by a first insulating member, and the second sealing electrode is preferably electrically insulated from the second jaw member by a second insulating member. The electrical connections preferably include a first connection capable of connecting one or both of the first and second sealing electrodes to the source of electrosurgical current, and a second connection capable of connecting one or both of the first and second arms to the source.

In one typical arrangement, the instrument operates as a monopolar electrosurgical instrument. In this arrangement, when the jaw members are in their closed position with tissue grasped therebetween, the instrument is capable of sealing the tissue by passing an electrosurgical current between one or both of the first and second sealing electrodes and a remote return electrode connected to the electrosurgical generator. When the jaw members are in their open position, the instrument is capable of cutting tissue by moving the instrument in a forward direction such that tissue comes into contact with the cutting electrode, an electrosurgical current passing from the cutting electrode to the remote return electrode. In this arrangement, the remote electrode acts as a return electrode for either the first and second sealing electrodes or for the cutting electrode, whichever is in use. Electrosurgical current passes through the patient from the electrodes to the remote return electrode, which is typically attached to the patient at a part of the body well away from the part of the body where the electrosurgical instrument is being used.

Alternatively, the instrument operates as a bipolar electrosurgical instrument. In this arrangement, the electrical connections comprise first, second and third connections capable of connecting the first sealing electrode, the second sealing electrode and the cutting electrode respectively to the source of electrosurgical current. In this bipolar arrangement, when the jaw members are in their closed position with tissue grasped therebetween, the instrument is capable of sealing the tissue by passing an electrosurgical current between the first and second sealing electrodes. Similarly when the jaw members are in their open position, the instrument is capable of cutting tissue by moving the instrument in a forward direction such that tissue comes into contact with the cutting electrode, an electrosurgical current passing between the cutting electrode and one or both of the first and second sealing electrodes. In this bipolar construction, when the instrument is being used to seal tissue, one of the sealing electrodes acts as the return electrode for the other sealing electrode. Alternatively, when the instrument is being used to cut tissue, one or both of the sealing electrodes act as the return electrode for the cutting electrode. In this way, electrosurgical current passes through the patient only at the surgical site, between the electrodes present on the instrument itself.

Embodiments of the invention further reside in a method of cutting tissue comprising the steps of

i) providing an electrosurgical instrument including
a handle including an actuating mechanism movable between a first position and a second position,
a pair of opposing first and second jaw members, movement of the actuating mechanism from its first position to its second position causing at least one of the jaw members to move relative to the other from a first open position in which the jaw members are disposed in a spaced relation relative to one another, to a second closed position in which the jaw members cooperate to grasp tissue therebetween, the first and second jaw members defining a throat area at which the jaw members converge when in their open position,
at least a first sealing electrode located on the first jaw member,
a flexible cutting electrode located adjacent the throat area mechanism and in fixed relationship thereto,

ii) maneuvering the instrument such that the first and second jaw members are in their open position with tissue to be cut located between the jaw members,

iii) supplying electrosurgical energy to the electrodes such that an electrosurgical cutting voltage is provided between the cutting electrode and the sealing electrode, and

iv) moving the instrument forwardly while maintaining the jaws in their open position and with the cutting electrode remaining stationary relative to the first and second jaw members so as to cut tissue coming into contact with the cutting electrode.

### BRIEF DESCRIPTION OF THE DRAWINGS

Embodiments of the invention will now be further described, by way of example only, with reference to the accompanying drawings, in which:

FIG. 1 is a schematic side view of an electrosurgical instrument in accordance with an embodiment of the present invention,

FIG. 2 is an enlarged perspective view of a part of the electrosurgical instrument of FIG. 1,

FIG. 3 is a schematic side view of an electrosurgical system including the instrument of FIG. 1,

FIG. 4 is a schematic side view of an alternative electrosurgical system including the instrument of FIG. 1, and

FIG. 5 is an enlarged perspective view of a part of an alternative embodiment of electrosurgical instrument in accordance with the present invention.

### DESCRIPTION OF THE EMBODIMENTS

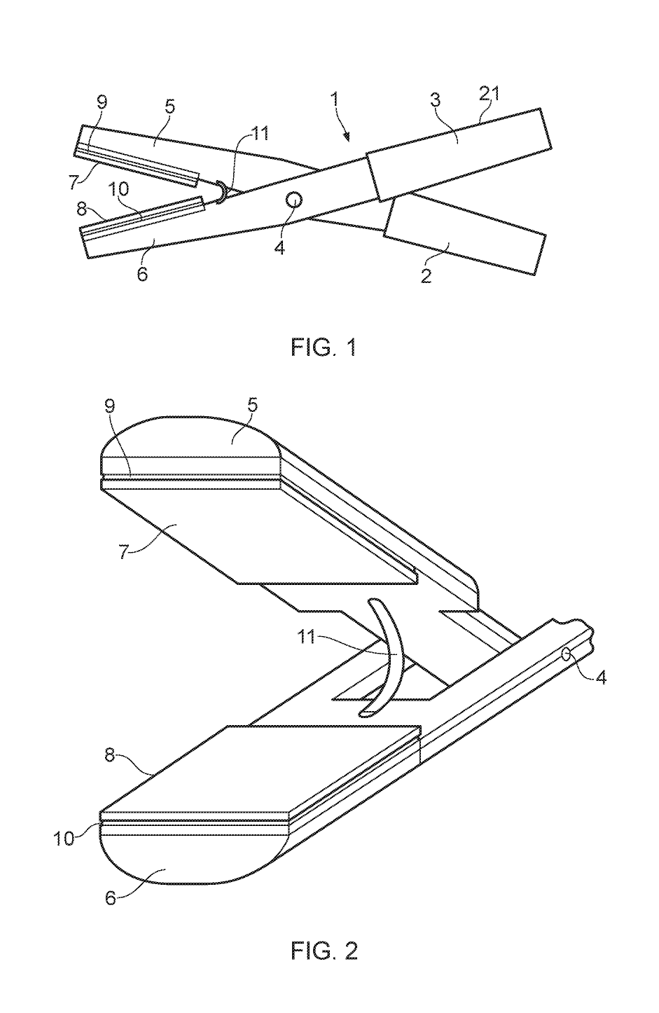

Referring to FIG. 1, an electrosurgical forceps instrument is shown generally at 1, and comprises a first arm 2 pivotably connected to a second arm 3 at a pivot point 4. The area of the first arm distal of the pivot point 4 constitutes a first jaw member 5, while the area of the second arm distal of the pivot point 4 constitutes a second jaw member 6. A first sealing electrode 7 is attached to the first jaw member 5, while a second sealing electrode 8 is attached to the second jaw member 6, in opposite corresponding relationship to the sealing electrode 7. The electrodes 7 & 8 are electrically insulated from the jaw members on which they are mounted by insulating members 9 & 10. An insulating sleeve 21 is provided on the arms 2 & 3 in the area held by the user of the instrument in order to protect them from any electrosurgical currents.

FIG. 2 shows the jaw members 5 & 6 in more detail. A cutting electrode in the form of a wire 11 is provided in the area adjacent the pivot point 4, the wire 11 being attached to and running between the first and second jaw members 5 & 6. In use, when the instrument 1 is required to seal tissue, the jaw members 5 & 6 are closed grasping tissue to be sealed between the first and second sealing electrodes 7 & 8. The electrosurgical coagulation of the tissue is preformed in a conventional manner, either in a bipolar arrangement with current passing from one sealing electrode to the other, or in a monopolar arrangement with current passing from one or both sealing electrodes to a remote return pad (not shown).

When the instrument 1 is required to cut tissue, the jaw members 5 & 6 are held in an open configuration as shown in FIGS. 1 & 2, and an electrosurgical cutting voltage is supplied to the wire 11. The instrument is then moved forwardly so that tissue comes into contact with the wire 11. As the tissue is cut by the wire 11, the instrument continues to be moved forwardly, continuing to cut tissue in a “running cut”, akin to cutting wrapping paper with an open pair of scissors.

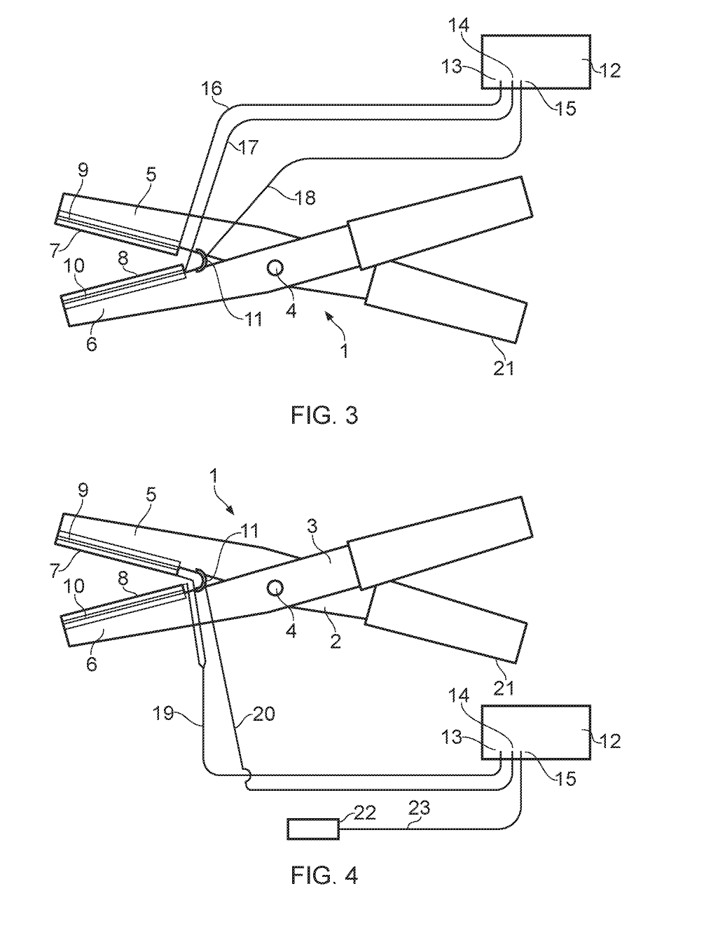

FIG. 3 shows the instrument 1 being connected to an electrosurgical generator 12 in a bipolar method of operation, the generator 12 having first second and third output connections 13, 14 & 15. In this arrangement, the first sealing electrode 7 is connected to the first output connection 13 of the generator by means of lead 16. Similarly, the second sealing electrode 8 is connected to the second output connection 14 of the generator by means of lead 17. Finally, the cutting wire 11 is connected to the third output connection 15 of the generator by means of lead 18. When the sealing of tissue is required, the generator 12 supplies a coagulating RF voltage between output connections 13 & 14, and hence first and second sealing electrodes 7 & 8. Conversely, when the cutting of tissue is required, the generator 12 supplies a cutting RF voltage between output connection 15 and output connections 13 & 14, and hence between the cutting wire 11 and one or both of the sealing electrodes 7 & 8.

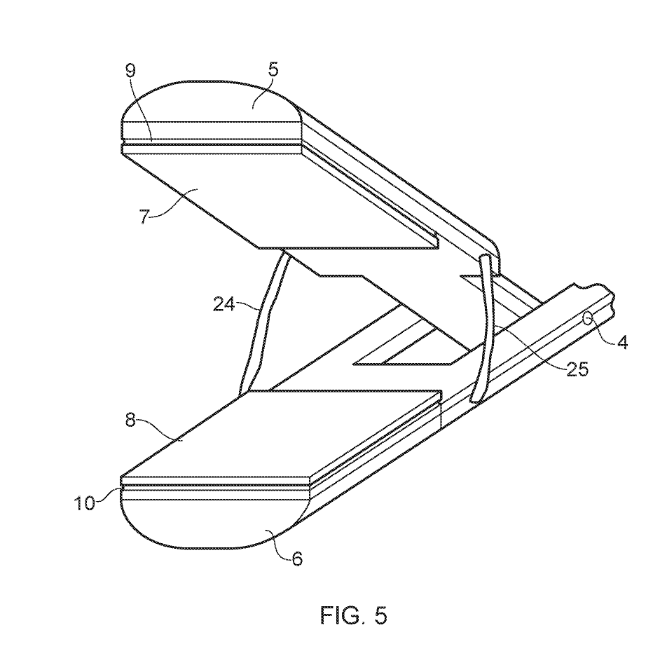

FIG. 4 shows the instrument 1 being connected to an electrosurgical generator 12 in a monopolar method of operation, the generator 12 once again having first second and third output connections 13, 14 & 15. In this arrangement, both the first and second sealing electrodes 7 & 8 are connected to the first output connection 13 of the generator by means of lead 19. The cutting wire 11 is connected to the second output connection 14 of the generator by means of lead 20. Finally, a remote return pad 22 is connected to the third output connection 15 of the generator by means of lead 23. When the sealing of tissue is required, the generator 12 supplies a coagulating RF voltage between output connections 13 & 15, and hence between the first and second sealing electrodes 7 & 8 and the return pad 22. Conversely, when the cutting of tissue is required, the generator 12 supplies a cutting RF voltage between output connection 14 and output connection 15, and hence between the cutting wire 11 and the return pad 22. Whether in sealing mode or in cutting mode, current flows from the instrument 1 to the return pad 22, which is attached to the body of the patient at an area remote from the area where the tissue is being treated.

FIG. 5 shows an alternative embodiment of instrument in which the single central wire 11 is replaced with a pair of wires 24 & 25, one located on each side of the jaw members 5 & 6. As before, the wires 24 & 25 run between the jaw members, in an area proximal of the sealing electrodes 7 & 8. The operation of the instrument of FIG. 5 is as previously described, with the wires 24 & 25 acting as cutting electrodes as the instrument 1 is moved forwardly with the jaw members 5 & 6 maintained in an open condition.

1. An electrosurgical instrument including
a handle including an actuating mechanism movable between a first position and a second position,
a pair of opposing first and second jaw members, movement of the actuating mechanism from its first position to its second position causing at least one of the jaw members to move relative to the other from a first open position in which the jaw members are disposed in a spaced relation relative to one another, to a second closed position in which the jaw members cooperate to grasp tissue therebetween, the first and second jaw members defining a throat area at which the jaw members converge when in their open position,
a first sealing electrode located on the first jaw member,
a second sealing electrode located on the second jaw member,
a flexible cutting electrode located adjacent the throat area and in fixed relationship thereto, and
electrical connections capable of connecting the instrument to a source of electrosurgical current, such that when the jaw members are in their closed position with tissue grasped therebetween, the instrument is capable of sealing the tissue by passing an electrosurgical current into the tissue from the first and second sealing electrodes, and when the jaw members are in their open position the instrument is capable of cutting tissue by moving the instrument in a forward direction such that tissue comes into contact with the cutting electrode, an electrosurgical current passing into the tissue from the cutting electrode.

2. An electrosurgical instrument according to claim 1, wherein the cutting electrode comprises a wire extending from the first jaw member to the second jaw member.

3. An electrosurgical instrument according to claim 2, wherein the wire is located on the longitudinal axis on the centre line of the first and second jaw members.

4. An electrosurgical instrument according to claim 2, wherein the cutting electrode comprises two wires extending from the first jaw member to the second jaw member, one on the outer face of each of the first and second jaw members.

5. An electrosurgical instrument according to claim 1, wherein the handle includes first and second pivoting arms, a distal portion of the first arm constituting the first jaw member and a distal portion of the second arm constituting the second jaw member.

6. An electrosurgical instrument according to claim 1, wherein the first sealing electrode is electrically insulated from the first jaw member by a first insulating member, and the second sealing electrode is electrically insulated from the second jaw member by a second insulating member.

7. An electrosurgical instrument according to claim 1, wherein the electrical connections include a first connection capable of connecting one or both of the first and second sealing electrodes to a source of electrosurgical current, and a second connection capable of connecting one or both of the first and second arms to a source of electrosurgical current.

8. An electrosurgical instrument according to claim 7, wherein the arrangement is such that when the jaw members are in their closed position with tissue grasped therebetween, the instrument is capable of sealing the tissue by passing an electrosurgical current between one or both of the first and second sealing electrodes and a remote return electrode connected to the source, and when the jaw members are in their open position the instrument is capable of cutting tissue by moving the instrument in a forward direction such that tissue comes into contact with the cutting electrode, an electrosurgical current passing from the cutting electrode to the remote return electrode.

9. An electrosurgical instrument according to claim 1, wherein the electrical connections comprise first, second and third connections capable of connecting the first sealing electrode, the second sealing electrode and the cutting electrode respectively to a source of electrosurgical current.

10. An electrosurgical instrument according to claim 9, wherein the arrangement is such that when the jaw members are in their closed position with tissue grasped therebetween, the instrument is capable of sealing the tissue by passing an electrosurgical current between the first and second sealing electrodes, and when the jaw members are in their open position the instrument is capable of cutting tissue by moving the instrument in a forward direction such that tissue comes into contact with the cutting electrode, an electrosurgical current passing between the cutting electrode and one or both of the first and second sealing electrodes.

11. A method of cutting tissue comprising the steps of
i) providing an electrosurgical instrument including
a handle including an actuating mechanism movable between a first position and a second position,
a pair of opposing first and second jaw members, movement of the actuating mechanism from its first position to its second position causing at least one of the jaw members to move relative to the other from a first open position in which the jaw members are disposed in a spaced relation relative to one another, to a second closed position in which the jaw members cooperate to grasp tissue therebetween, the first and second jaw members defining a throat area at which the jaw members converge when in their open position,
at least a first sealing electrode located on the first jaw member,
a flexible cutting electrode located adjacent the throat area and in fixed relationship thereto,
ii) maneuvering the instrument such that the first and second jaw members are in their open position with tissue to be cut located between the jaw members,
iii) supplying electrosurgical energy to the electrodes such that an electrosurgical cutting voltage is provided between the cutting electrode and the sealing electrode, and
iv) moving the instrument forwardly while maintaining the jaws in their open position and with the cutting electrode remaining stationary relative to the first and second jaw members so as to cut tissue coming into contact with the cutting electrode.
